(19) United States
(12) Patent Application Publication
(10) Pub. No.: US 2024/0343861 A1
(43) Pub. Date: Oct. 17, 2024

(54) Food and Medical Grade Polymer Composition and Articles Made Therefrom

(71) Applicant: Celanese International Corporation, Irving, TX (US)

(72) Inventors: Qamer Zia, Raunheim (DE); Kirsten Markgraf, Weinheim (DE); Dirk Zierer, Hattersheim (DE); Patrick Nickolay, Villmar (DE); Peter Feist, Florence, KY (US)

(21) Appl. No.: 18/632,730

(22) Filed: Apr. 11, 2024

Related U.S. Application Data
(60) Provisional application No. 63/495,672, filed on Apr. 12, 2023.

(51) Int. Cl. C08G 63/183; C08K 5/3492; C08K 7/14

(52) U.S. Cl. CPC C08G 63/183; C08K 5/34928; C08K 7/14

(57) ABSTRACT

A fiber reinforced polyester polymer composition is disclosed that is specially formulated for food contact applications and/or medical applications. The polyester polymer composition contains glass fibers that are either bisphenol-A free or contain free bisphenol-A in only trace/residual amounts. The composition can be formulated without using conventional stabilizers, antioxidants and/or lubricants. The polymer composition displays excellent mechanical properties.

### RELATED APPLICATIONS

The present application is based upon and claims priority to U.S. Provisional Application Ser. No. 63/495,672, having a filing date of Apr. 12, 2023, which is incorporated herein by reference.

### BACKGROUND

Engineering thermoplastics are often used in numerous and diverse applications in order to produce molded parts and products. For instance, thermoplastic polymers are used to produce all different types of molded products, such as injection molded products, blow molded products, and the like. Thermoplastic polymers, for instance, can be formulated in order to be chemically resistant, to have excellent strength properties and, when formulating compositions containing elastomers, to be flexible. Of particular advantage, many polymers can be melt processed due to their thermoplastic nature. In addition, many polymers can be recycled and reprocessed.

One objective in producing molded parts from thermoplastic polymers is to produce parts with desired mechanical properties. These mechanical properties can include a combination of tensile strength and impact resistance. When producing polyester polymer compositions, for instance, various additives can be blended with the polyester polymer in order to improve one or more properties. For example, glass fibers are typically added in order to improve strength. In addition, various stabilizers are combined with the polymers so that the strength characteristics do not degrade over time.

Another objective in producing molded parts from thermoplastic polymers is the ability to quickly mold the parts and increase productivity. Each polymer formulation, for instance, can present a unique set of problems related to the melt processing characteristics of the composition which can create longer cycle times, cause mold deposits to form, and/or negatively impact the ability to remove the part from a mold. In order to reduce cycle times and in order to improve the stability of the polymer composition during molding, polyester polymer compositions are typically combined with various other additives including lubricants, waxes, and various other stabilizers.

Many of the additives that are added to polymer compositions, particularly polyester polymer compositions, however, are not approved for various different food handling applications and medical applications. For example, glass fibers are typically coated with a sizing agent that can contain one or more chemicals not approved for food contact and/or medical applications by many governments. Thus, problems have been experienced in formulating polyester polymer compositions that have the necessary mechanical properties while still being suitable for producing parts that are used in food contact applications and/or medical applications. Consequently, a need currently exists for a polyester polymer formulation that is approved for food handling applications and/or medical applications while still having a desired blend of mechanical properties and/or polymer processing properties.

### SUMMARY

In general, the present disclosure is directed to a polyester polymer composition, particularly a composition containing a polybutylene terephthalate polymer, that is particularly well suited for food contact applications. The present disclosure is also directed to a polyester polymer composition that can also be used to produce various medical products. The polymer composition of the present disclosure is particularly formulated to have a blend of mechanical properties without containing many conventional additives that in the past were thought necessary to achieve the strength and impact resistance properties needed for the particular applications.

In one embodiment, the present disclosure is directed to a food and medical grade polymer composition. The polymer composition includes a polyester polymer comprising a polybutylene terephthalate polymer. The polyester polymer is present in the composition in an amount greater than about 40% by weight, such as in an amount greater than about 50% by weight, such as in an amount greater than about 55% by weight, such as in an amount greater than about 60% by weight. The polymer composition further contains a mineral nucleant. The mineral nucleant can be present in the composition in an amount less than about 0.8% by weight, such as in an amount less than about 0.6% by weight, such as in an amount less than about 0.4% by weight, and generally greater than about 0.01% by weight. The polymer composition further contains reinforcing fibers comprising glass fibers. The reinforcing fibers are present in the polymer composition in an amount from about 3% to about 50% by weight, such as in an amount from about 15% to about 45% by weight, such as in an amount from about 25% to about 35% by weight. In accordance with one embodiment of the present disclosure, the reinforcing fibers contain free (not chemically bound) bisphenol-A in an amount less than about 200 ppb, such as less than about 100 ppb, such as less than about 60 ppb, such as in an amount less than about 45 ppb, such as in an amount less than about 30 ppb, such as in an amount less than about 20 ppb, such as in an amount less than about 10 ppb, such as in an amount less than about 5 ppb. In one embodiment, the glass fibers incorporated into the polymer composition are bisphenol-A free.

The polymer composition of the present disclosure can be formulated without containing various conventional additives and while still having excellent mechanical properties. For example, the polymer composition can be formulated to not contain any antioxidants, particularly hindered phenolic antioxidants. The composition can also be formulated to not contain any heat stabilizers, such as diphosphite stabilizers. In addition, the polymer composition can be formulated to be free of lubricants and waxes. Thus, in one embodiment, the polybutylene terephthalate polymer, the glass fibers, and the mineral nucleant can account for greater than about 95% by weight of the composition, such as greater than about 97% by weight of the composition, such as greater than about 99% by weight of the composition.

In one embodiment, glass fibers having a relatively small diameter can be used. For instance, the glass fibers can have an average diameter of less than about 12 microns, such as less than about 11 microns, and generally greater than about 3 microns, such as greater than about 5 microns.

The mineral nucleant, in one embodiment, can be talc. The mineral nucleant or talc can be uncoated and can have a median particle size of less than about 4 microns, such as less than about 3 microns, such as less than about 2.5 microns. In one embodiment, microcrystalline talc particles are used that are at least about 90% by weight pure, such as at least about 95% by weight pure.

The polymer composition of the present disclosure can display excellent mechanical properties. For instance, the polymer composition can display a tensile strength at break of greater than about 135 MPa, such as greater than about 140 MPa, such as greater than about 145 MPa, such as greater than about 150 MPa. The polymer composition can display a tensile modulus of greater than about 9,500 MPa, such as greater than about 9,750 MPa, such as greater than about 9,900 MPa. In addition, the polymer composition can display a notched Charpy impact strength of greater than about 9 kJ/m², such as greater than about 9.2 kJ/m².

Various different molded articles can be made in accordance with the present disclosure for use in numerous applications. In one embodiment, polymer articles can be formed from the polymer composition using injection molding. In one embodiment, the polymer composition can be used to form at least one component of a baby bottle. In an alternative embodiment, the polymer composition can be used to form at least one component of a medical product. The medical product, for instance, can be an injector or an inhaler.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a perspective view of a baby bottle that can include a component made in accordance with the present disclosure;

FIG. 2 is a perspective view of a handle for a consumer appliance and/or a food handling device that can include a component made in accordance with the present disclosure;

FIG. 3 is a perspective view of a medical inhaler made in accordance with the present disclosure; and

FIG. 4 is a side view of a medical injector that may be made in accordance with the present disclosure.

Repeat use of reference characters in the present specification and drawings is intended to represent the same or analogous features or elements of the present invention.

### Definition

The following is the procedure for determining “free” bisphenol-A in a sample, such as a sample of a compounded polymer composition, in accordance with the present disclosure. Bisphenol-A in a sample can exist in a free form and in a bound form (e.g. chemically bound). The bound form, for instance, can be present in epoxy resins or in polycarbonates. In conducting an analysis of free bisphenol-A in a sample, false positives can occur if the bound bisphenol-A becomes free bisphenol-A during sample preparation, such as by dissolving the sample in a solvent. The following is a procedure that only measures free bisphenol-A in accordance with the present disclosure.

Extraction can be performed on glass fiber or on cryomilled glass fiber reinforced polymer (e.g. polybutylene terephthalate). Ten (10) grams of sample is extracted via Soxhlet extraction in 50 mL methanol at 55° C. for 48 hours. The resulting slurry is filtered using Whatman paper filter, and an aliquot is taken for analysis. Samples are dried down at 60° C. The extract is resuspended in 5 mL methanol and high-resolution mass spectrometry is performed (UHPLC-HRMS).

### UHPLC-HRMS Experimental Conditions:

System: Thermo Vanquish UPLC coupled to Q-Exactive Plus (LC-MS 2)
Column: Phenomenex Kinetex 2.1×100 mm, 2.6 μm
Column Temperature: 35° C.
Injection Volume: 20 μL
Flow Rate: 0.300 mL/min
Detector Wavelength: 210 nm; full scan 190 nm-300 nm
Detector bandwidth: 10 nm
Free-BPA results in mg/kg of sample.
*Detection limit ˜0.1 ppm.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a thermoplastic polymer composition and to polymer articles made from the composition that not only have excellent strength properties but are also specially formulated for food contact applications and/or medical applications. In general, the polymer composition of the present disclosure contains a glass-reinforced polyester polymer, particularly a polybutylene terephthalate polymer. Particular glass fibers are selected that are covered with a sizing agent particularly formulated for meeting various governmental regulations with respect to use in food contact applications. For example, in the past, conventionally used sizing agents for glass fibers contained bisphenol-A. The glass fibers selected for use in the present disclosure, however, are either free from bisphenol-A or contain free bisphenol-A in very minor amounts, such as in amounts less than 100 ppb, such as less than about 65 ppb. In one aspect, the glass fibers are also selected that do not contain components made from bisphenol-A, e.g. polycarbonate or epoxy resins, which could liberate bisphenol-A upon degradation or chemical reaction during processing.

In addition to using particular types of reinforcing fibers, the formulation of the present disclosure can also be free of conventional stabilizers and processing aids, such as lubricants. In order to improve molding cycle times or other properties, the polymer composition contains a mineral nucleant that is also approved for food contact applications. Through the use of particular ingredients and in particular amounts, polymer compositions can be produced having excellent mechanical properties, particularly a combination of strength and impact resistance.

For example, polymer compositions formulated in accordance with the present disclosure can display a tensile modulus, when tested according to ISO Test 527, of greater than about 9,500 MPa, such as greater than about 9,750 MPa, such as greater than about 9,900 MPa, and generally less than about 20,000 MPa.

The tensile strength at break of the polymer composition can also be relatively high. For instance, the tensile strength at break can be greater than about 135 MPa, such as greater than about 140 MPa, such as greater than about 145 MPa, such as greater than about 150 MPa, and generally less than about 300 MPa.

In addition to excellent tensile strength properties, the polymer composition can also display excellent impact resistance. For instance, the polymer composition can exhibit a Charpy notched impact strength when tested at 23° C. of greater than about 9 kJ/m², such as greater than about 9.2 kJ/m², such as greater than about 9.3 kJ/m², and generally less than about 20 kJ/m².

Even in view of the above physical and mechanical properties, polymer compositions made according to the present disclosure can still be formulated so as to be completely safe for food contact and medical applications without containing many conventional ingredients that were thought necessary in the past. In this regard, the polymer composition of the present disclosure can be formulated so that every component contained in the composition meets governmental regulations regarding food handling or medical applications. For example, every component contained in the polymer composition can be approved for use according to the United States Food and Drug Administration food contact standards and approved listings as found in Title 21 of the Code of Federal Regulations (as in existence in March of 2021). For example, each polymer contained within the polymer composition can be approved for food handling applications as indicated in 21 CFR 177. Each component contained in the polymer composition can also be approved for food handling applications according to 21 CFR 174.

Each component contained within the polymer composition can also meet or exceed all food contact standards such as Regulation (EC) No. 1935/2004, 2023/2006, 10/2011, Resolution AP (89) 1, Germany BfR IX, Spain Real Decreto 847/2011, and Italy Decreto 21/3/73; and Chinese food contact standards such as GB 9685-2016.

In one embodiment, the polymer composition of the present disclosure contains one or more polyester polymers in combination with glass fibers covered with a specifically selected sizing composition and a particularly selected mineral nucleant. In one embodiment, the polyester polymer selected for use in the present disclosure is a polybutylene terephthalate polymer. The polybutylene terephthalate polymer can be bio-based. In one aspect, the polybutylene terephthalate polymer, the glass fibers, and the mineral nucleant can account for more than 95% by weight of the polymer composition, such as by more than about 97% by weight of the polymer composition, such as by more than 99% by weight of the polymer composition. As described above, the polymer composition can be formulated without containing many conventional additives including antioxidants, stabilizers, processing aids, waxes, lubricants, and the like. In addition, the polymer composition can be formulated without containing any isocyanates, epoxy resins, carbodiimides, or the like.

The thermoplastic polymer used as the matrix polymer to form molded articles in accordance with the present disclosure can vary depending upon the particular application and the desired result. Thermoplastic polymers that may be used in accordance with the present disclosure include, for instance, one or more polyester polymers. The matrix polymer, for instance, can be a polybutylene terephthalate polymer alone or in combination with a polyethylene terephthalate polymer. In other embodiments, the thermoplastic polymer can comprise a polyamide polymer, a polyoxymethylene polymer, or mixtures thereof.

As described above, in one embodiment, the thermoplastic matrix polymer contained in the polymer composition comprises one or more polyester polymers. The polyester polymer generally comprises a polyalkylene terephthalate polymer.

Polyalkylene terephthalate polymers suitable for use herein are derived from an aliphatic or cycloaliphatic diol, or mixtures thereof, containing from 2 to about 10 carbon atoms and an aromatic dicarboxylic acid.

The polyesters which are derived from a cycloaliphatic diol and an aromatic dicarboxylic acid are prepared by condensing either the cis- or trans-isomer (or mixtures thereof) of, for example, 1,4-cyclohexanedimethanol with the aromatic dicarboxylic acid.

Examples of aromatic dicarboxylic acids include isophthalic or terephthalic acid, 1,2-di(p-carboxyphenyl)ethane, 4,4′-dicarboxydiphenyl ether, etc., and mixtures of these. All of these acids contain at least one aromatic nucleus. Fused rings can also be present such as in 1,4- or 1,5- or 2,6-naphthalene-dicarboxylic acids. In one embodiment, the dicarboxylic acid is terephthalic acid or mixtures of terephthalic and isophthalic acid.

In one embodiment, the polyalkylene terephthalate polymer present in the polymer composition comprises a polybutylene terephthalate polymer. For example, the polymer composition may contain a polybutylene terephthalate polymer in an amount greater than about 40% by weight, such as in an amount greater than about 45% by weight, such as in an amount greater than about 50% by weight, such as in an amount greater than about 55% by weight, such as in an amount greater than about 60% by weight. The polybutylene terephthalate polymer is generally present in an amount less than about 90% by weight, such as in an amount less than about 80% by weight.

The melt flow rate of the polyester polymer, such as the polybutylene terephthalate polymer, can vary depending upon the particular application and the other ingredients contained in the composition. In general, the melt flow rate can be from about 20 cm³/10 min to about 120 cm³/10 min. In one particular application, the melt flow rate of the polyester polymer or polybutylene terephthalate polymer is from about 50 cm³/10 min to about 80 cm³/10 min. In an alternative embodiment, the melt flow rate of the polyester polymer can be from about 25 cm³/10 min to about 50 cm³/10 min. As used herein, melt flow rate of the polyester polymer is determined at a temperature of 250° C. and at a load of 2.16 kg according to ISO Test 1133.

The polymer composition may contain the polybutylene terephthalate polymer alone or in combination with other thermoplastic polymers. For instance, the polybutylene terephthalate polymer may be combined with other polyester polymers and/or a polycarbonate polymer. Other polyester polymers that may be present in the composition include a polyethylene terephthalate polymer or a polyethylene terephthalate copolymer. For instance, a polyethylene terephthalate copolymer or modified polyethylene terephthalate polymer can be produced with a modifying acid or a modifying diol.

As used herein, the terms “modifying acid” and “modifying diol” are meant to define compounds, which can form part of the acid and diol repeat units of a polyester, respectively, and which can modify a polyester to reduce its crystallinity or render the polyester amorphous. In one embodiment, however, the polyesters present in the polymer composition of the present disclosure are non-modified and do not contain a modifying acid or a modifying diol.

Examples of modifying acid components may include, but are not limited to, isophthalic acid, phthalic acid, 1,3-cyclohexanedicarboxylic acid, 1,4-cyclohexane dicarboxylic acid, 2,6-naphthaline dicarboxylic acid, succinic acid, glutaric acid, adipic acid, sebacic acid, suberic acid, 1,12-dodecanedioic acid, and the like. In practice, it is often preferable to use a functional acid derivative thereof such as the dimethyl, diethyl, or dipropyl ester of the dicarboxylic acid. The anhydrides or acid halides of these acids also may be employed where practical. Preferred is isophthalic acid.

Examples of modifying diol components may include, but are not limited to, neopentyl glycol, 1,4-cyclohexanedimethanol, 1,2-propanediol, 1,3-propanediol, 2-Methy-1,3-propanediol, 1,4-butanediol, 1,6-hexanediol, 1,2-cyclohexanediol, 1,4-cyclohexanediol, 1,2-cyclohexanedimethanol, 1,3-cyclohexanedimethanol, 2,2,4,4-tetramethyl 1,3-cyclobutane diol, Z,8-bis(hydroxymethyltricyclo-[5.2.1.0]-decane wherein Z represents 3, 4, or 5; 1,4-Bis(2-hydroxyethoxy)benzene, and diols containing one or more oxygen atoms in the chain, e.g. diethylene glycol, triethylene glycol, dipropylene glycol, tripropylene glycol, and the like. In general, these diols contain 2 to 18, preferably 2 to 8 carbon atoms. Cycloalphatic diols can be employed in their cis or trans configuration or as mixtures of both forms.

When present, the polyester polymer combined with the polybutylene terephthalate can be added to the polymer composition in amounts generally greater than about 3% by weight, such as in amounts greater than about 5% by weight, such as in amounts greater than about 8% by weight. The polyester polymer is generally present in an amount less than about 25% by weight, such as in an amount less than about 20% by weight, such as in an amount less than about 15% by weight, such as in an amount less than about 12% by weight.

In one embodiment, the polybutylene terephthalate polymer contained in the polymer composition comprises a bio-based polyester polymer. For instance, the polybutylene terephthalate polymer can be formed from a butane diol that was produced from a renewable bio-source. For instance, the butane diol can be produced from bio-mass or from a bio-gas. In one embodiment, for instance, bio-mass can be used to produce ethanol or methanol which is then converted into butane diol for producing the polyester polymer.

The polymer composition also contains reinforcing fibers in addition to the thermoplastic polymer matrix. Thermoplastic polymers, such as polyester polymers, are combined with fibrous fillers in order to increase the modulus and/or tensile strength of parts and products made from the reinforced composition. In the past, however, reinforcing fibers, particularly glass fibers, were coated with a sizing agent that included various components that have come under governmental scrutiny when used in food handling and/or medical applications.

The sizing treatment was applied to the glass fibers in order to produce or process the fibers and/or in order to improve the adhesion of the fibers to the thermoplastic polymer matrix. Sizing compositions used in the past, for instance, may contain silanes, film forming agents, lubricants, wetting agents, adhesive agents optionally antistatic agents and plasticizers, emulsifiers and optionally further additives. Specific examples of silanes are aminosilanes, e.g. 3-trimethoxysilylpropylamine, N-(2-aminoethyl)-3-aminopropyltrimethoxy-silane, N-(3-trimethoxysilanylpropyl)ethane-1,2-diamine, 3-(2-aminoethyl-amino)propyltrimethoxysilane, N-[3-(trimethoxysilyl)propyl]-1,2-ethane-diamine.

The glass fibers incorporated into the polymer composition of the present disclosure, however, can include a specially formulated sizing composition that does not contain many of the conventional ingredients used in the past. In particular, past formulations contained free bisphenol-A and/or contained components derived from bisphenol-A, such as epoxy resins or polycarbonates, that was thought to be a necessary or essential part of the sizing composition.

In one aspect, the glass fibers used in accordance with the present disclosure and the resulting compounded polymer composition, however, contain free (chemically not bound) bisphenol-A in an amount less than about 2 ppm, such as in an amount less than about 1.6 ppm, such as in an amount less than about 200 ppb, such as less than about 100 ppb, such as less than about 65 ppb, such as less than about 55 ppb, such as less than about 45 ppb, such as less than about 35 ppb, such as less than about 25 ppb, such as less than about 15 ppb, such as less than about 10 ppb, such as less than about 5 ppb, such as less than about 2 ppb. In one embodiment, the glass fibers incorporated into the polymer composition of the present disclosure are free of bisphenol-A.

The polymer composition may contain some chemically bound bisphenol-A. For example, the polymer composition can contain a total amount of bisphenol-A (free+bound) in an amount less than about 5 ppm, such as in an amount less than about 3 ppm, such as in an amount less than about 2 ppm, such as in an amount less than about 500 ppb, such as in an amount less than about 250 ppb, such as in an amount less than about 165 ppb, such as in an amount less than about 130 ppb, such as in an amount less than about 100 ppb, such as in an amount less than about 80 ppb, such as in an amount less than about 60 ppb, such as in an amount less than about 40 ppb, such as in an amount less than about 20 ppb, such as in an amount less than about 10 ppb.

In various embodiments, the sizing composition applied to the glass fibers can also be free of silanes and can be made from various other components. For example, in one embodiment, the glass fibers are coated with a food grade polymer. In one particular embodiment, the food grade polymer can be a polyurethane polymer.

The reinforcing fibers may be compounded into the polymer matrix, for example in an extruder or kneader. Glass fibers which are suitable for the molding composition of the present disclosure can be obtained from Johns Manville, OCV, Nippon Electric Glass Co.

Fiber diameters can vary depending upon the particular fiber used and whether the fiber is in either a chopped or a continuous form. In one aspect, it was discovered that using fibers having a relatively small average diameter can improve the mechanical properties of the polymer composition when coated with the sizing composition containing small amounts of bisphenol-A. For instance, the average diameter of the glass fibers can be less than about 17 microns, such as less than about 14 microns, such as less than about 13 microns, such as less than about 12 microns, and generally greater than about 3 microns, such as greater than about 6 microns, such as greater than about 8 microns.

In general, reinforcing fibers can be present in the polymer composition in amounts sufficient to increase the tensile strength of the composition. The reinforcing fibers, for example, can be present in the polymer composition in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. The reinforcing fibers are generally present in an amount less than about 55% by weight, such as in an amount less than about 50% by weight, such as in an amount less than about 45% by weight, such as in an amount less than about 40% by weight, such as in an amount less than about 35% by weight.

The polymer composition of the present disclosure can also contain a mineral nucleant. The nucleant can be selected from the group consisting of alkali metal salts having anions which are oxides of the elements from Group IV of the Periodic Table; barium sulfate; and talc.

In one embodiment, the mineral nucleant is talc. Talc (CAS No. 14807-96-6) is a sheet silicate having the chemical composition Mg₃[Si₄O₁₀(OH)₂], which, according to the polymorph, crystallizes as talc-1A in the triclinic crystal system or as talc-2M in the monoclinic crystal system. In one embodiment, talc present in the polymer composition is a microcrystalline talc having a relatively small particle size. Unlike when talc is used as a filler, the talc particles can be uncoated.

In one embodiment, microcrystalline talc having a median particle size d50 determined using a SediGraph in the range from 0.5 to 10 μm is used, such as in the range from 1.0 to 7.5 μm, such as in the range from 1.5 to 5.0 μm, such as in the range from 1.8 to 4.5 μm.

The particle size of the talc is determined by sedimentation in a fully dispersed state in an aqueous medium with the aid of a “Sedigraph 5100” as supplied by Micrometrics Instruments Corporation, Norcross, Ga., USA. The Sedigraph 5100 delivers measurements and a plot of cumulative percentage by weight of particles having a size referred to as “equivalent sphere diameter” (esd), minus the given esd values.

The median particle size d50 is the value determined from the particle esd at which 50% by weight of the particles have an equivalent sphere diameter smaller than this d50 value. The underlying standard is ISO 13317-3.

In one embodiment, microcrystalline talc is defined via the BET surface area. Microcrystalline talc for use in accordance with the present disclosure can have a BET surface area, which can be determined in analogy to DIN ISO 9277, in the range from 5 to 25 m²·g⁻¹, such as in the range from 10 to 18 m²·g⁻¹, such as in the range from 12 to 15 m²·g⁻¹.

The talc particles incorporated into the polymer composition can contain talc that has a purity of greater than 96% by weight, such as in an amount greater than 97% by weight, such as in an amount greater than 98% by weight, such as in an amount greater than 99% by weight. The talc particles can contain chlorite in an amount less than about 2% by weight, such as in an amount less than about 1% by weight, such as in an amount less than about 0.1% by weight, can contain dolomite in an amount less than about 2% by weight, such as in an amount less than about 1% by weight, such as in an amount less than about 0.1% by weight, and can contain magnesite in an amount less than about 2% by weight, such as in an amount less than about 1% by weight, such as in an amount less than about 0.5% by weight, such as in an amount less than about 0.1% by weight. In one embodiment, the talc particles can be free of chlorite, can be free of dolomite, can be free of magnesite, or can be free of all the above or at least two of the above.

The nucleant or talc particles can be present in the polymer composition in very relative amounts while still providing significant advantages and benefits. For instance, the nucleant can be present in the polymer composition in an amount less than about 1.5% by weight, such as in an amount less than about 0.8% by weight, such as in an amount less than about 0.6% by weight, such as in an amount less than about 0.4% by weight. The talc particles are generally present in the polymer composition in an amount greater than about 0.01% by weight, such as in an amount greater than about 0.05% by weight, such as in an amount greater than about 0.1% by weight.

As described above, the polymer composition of the present disclosure can be formulated so as to be free of many conventionally used additives and ingredients while still retaining an excellent balance of strength properties and impact resistance. In one aspect, the polyester polymer composition of the present disclosure can be formulated so as to be free of many conventionally used lubricants and mold release agents. For instance, the polymer composition can be formulated to be free of certain fatty acid esters, particularly fatty acid esters having relatively long carbon chains. In one particular embodiment, for instance, the composition of the present disclosure is free from any fatty acid esters that are derived from montanic acids, such as esters of a montanic acid in combination with a polyol. The composition, for example, can be free of a mixture of montanic acid esters and calcium montanate.

Other lubricants that can be excluded from the polymer composition or contained in very minor amounts include waxes, such as polyolefin waxes and amide waxes. Such waxes include ethylene bis stearamide wax, other bisamides, N-(2-hydroxyethyl)12-hydroxystearamide, and/or N,N′-(ethylene bis)12-hydroxystearamide.

The polymer composition can also optionally be free of specific antioxidants that were commonly used in the past. For example, in one embodiment, the polyester polymer composition of the present disclosure is free of hindered phenolic antioxidants or contains such antioxidants in very limited amounts. For example, the polymer composition can be formulated to contain hindered phenolic antioxidants in an amount less than about 0.1% by weight, such as in an amount less than about 0.05% by weight, such as in an amount less than about 0.03% by weight, such as in an amount of 0% by weight.

Examples of such phenolic antioxidants include, for instance, calcium bis(ethyl 3,5-di-tert-butyl-4-hydroxybenzylphosphonate) (Irganox® 1425); terephthalic acid, 1,4-dithio-, S,S-bis(4-tert-butyl-3-hydroxy-2,6-dimethylbenzyl) ester (Cyanox® 1729); triethylene glycol bis(3-tert-butyl-4-hydroxy-5-methylhydrocinnamate); hexamethylene bis(3,5-di-tert-butyl-4-hydroxyhydrocinnamate (Irganox® 259); 1,2-bis(3,5,di-tert-butyl-4-hydroxyhydrocinnamoyl)hydrazide (Irganox® 1024); 4,4′-di-tert-octyldiphenamine (Naugalube® 438R); phosphonic acid, (3,5-di-tert-butyl-4-hydroxybenzyl)-,dioctadecyl ester (Irganox® 1093); 1,3,5-trimethyl-2,4,6-tris(3′,5′-di-tert-butyl-4′ hydroxybenzyl)benzene (Irganox® 1330); 2,4-bis(octylthio)-6-(4-hydroxy-3,5-di-tert-butylanilino)-1,3,5-triazine (Irganox® 565); isooctyl 3-(3,5-di-tert-butyl-4-hydroxyphenyl)propionate (Irganox® 1135); octadecyl 3-(3,5-di-tert-butyl-4-hydroxyphenyl)propionate (Irganox® 1076); 3,7-bis(1,1,3,3-tetramethylbutyl)-1 OH-phenothiazine (Irganox® LO 3); 2,2′-methylenebis(4-methyl-6-tert-butylphenol)monoacrylate (Irganox® 3052); 2-tert-butyl-6-[1-(3-tert-butyl-2-hydroxy-5-methylphenyl)ethyl]-4-methylphenyl acrylate (Sumilizer® TM 4039); 2-[1-(2-hydroxy-3,5-di-tert-pentylphenyl)ethyl]-4,6-di-tert-pentylphenyl acrylate (Sumilizer® CS); 1,3-dihydro-2H-Benzimidazole (Sumilizer® MB); 2-methyl-4,6-bis[(octylthio)methyl]phenol (Irganox® 1520): N,N′-trimethylenebis-[3-(3,5-di-tert-butyl-4-hydroxyphenyl)propionamide (Irganox® 1019); 4-n-octadecyloxy-2,6-diphenylphenol (Irganox® 1063); 2,2′-ethylidenebis[4,6-di-tert-butylphenol](Irganox® 129); N N′-hexamethylenebis(3,5-di-tert-butyl-4-hydroxyhydrocinnamamide) (Irganox® 1098); diethyl (3,5-di-tert-butyl-4-hydroxybenxyl)phosphonate (Irganox® 1222); 4,4′-di-tert-octyldiphenylamine (Irganox® 5057); N-phenyl-1-napthalenamine (Irganox® L 05); tris[2-tert-butyl-4-(3-ter-butyl-4-hydroxy-6-methylphenylthio)-5-methyl phenyl]phosphite (Hostanox® OSP 1); zinc dinonyidithiocarbamate (Hostanox® VP-ZNCS 1); 3,9-bis[1,1-diimethyl-2-[(3-tert-butyl-4-hydroxy-5-methylphenyl)propionyloxy]ethyl]-2,4,8,10-tetraoxaspiro[5.5]undecane (Sumilizer® AG80); pentaerythrityl tetrakis[3-(3,5-di-tert-butyl-4-hydroxyphenyl)propionate] (Irganox® 1010); ethylene-bis(oxyethylene)bis[3-(5-tert-butyl-4-hydroxy-m-tolyl)-propionate (Irganox® 245); 3,5-di-tert-butyl-4-hydroxytoluene (Lowinox BHT, Chemtura) and the like.

Reducing or eliminating the use of hindered phenolic antioxidants may actually increase the bond strength between a molded article made from the polyester composition and an elastomeric material, particularly an elastomeric material formed from a copolyester elastomer. Although unknown, it is believed that hindered phenolic antioxidants may prevent ester interchange at the boundary layer between the molded article and the elastomeric material.

In addition to being free of phenolic antioxidants, the polymer composition of the present disclosure can also be formulated to be free of diphosphite stabilizers.

Another stabilizer that can excluded from the polyester composition are hindered amine light stabilizers (“HALS”). HALS compounds may be derived from a substituted piperidine, such as alkyl-substituted piperidyl, piperidinyl, piperazinone, alkoxypiperidinyl compounds, and so forth. For example, hindered amines may be derived from a 2,2,6,6-tetraalkylpiperidinyl.

Specific examples of hindered amines may include, for instance, bis-(2,2,6,6-tetramethyl-4-piperidyl) sebacate (Tinuvin® 770 from Ciba Specialty Chemicals, MW=481); bis-(1,2,2,6,6-pentamethyl-4-piperidinyl)-(3,5-ditert.butyl-4-hydroxybenzyl)butyl-propane dioate; bis-(1,2,2,6,6-pentamethyl-4-piperidinyl)sebacate; 8-acetyl-3-dodecyl-7,7,9,9-tetramethyl-1,3,8-triazaspiro-(4,5)-decane-2,4-dione, butanedioic acid-bis-(2,2,6,6-tetramethyl-4-piperidinyl) ester; tetrakis-(2,2,6,6-tetramethyl-4-piperidyl)-1,2,3,4-butane tetracarboxylate; 7-oxa-3,20-diazadispiro(5.1.11.2) heneicosan-20-propanoic acid, 2,2,4,4-tetramethyl-21-oxo, dodecyl ester; N-(2,2,6,6-tetramethyl-4-piperidinyl)-N′-amino-oxamide; o-t-amyl-o-(1,2,2,6,6-pentamethyl-4-piperidinyl)-monoperoxi-carbonate; β-alanine, N-(2,2,6,6-tetramethyl-4-piperidinyl), dodecylester; ethanediamide, N-(1-acetyl-2,2,6,6-tetramethylpiperidinyl)-N′-dodecyl; 3-dodecyl-1-(2,2,6,6-tetramethyl-4-piperidinyl)-pyrrolidin-2,5-dione; 3-dodecyl-1-(1,2,2,6,6-pentamethyl-4-piperidinyl)-pyrrolidin-2,5-dione; 3-dodecyl-1-(1-acetyl,2,2,6,6-tetramethyl-4-piperidinyl)-pyrrolidin-2,5-dione, (Sanduvar® 3058 from Clariant, MW=448.7); 4-benzoyloxy-2,2,6,6-tetramethylpiperidine; 1-[2-(3,5-di-tert-butyl-4-hydroxyphenylpropionyloxy)ethyl]-4-(3,5-di-tert-butyl-4-hydroxylphenyl propionyloxy)-2,2,6,6-tetramethyl-piperidine; 2-methyl-2-(2″,2″,6″,6″-tetramethyl-4″-piperidinylamino)-N-(2′,2′,6′6′-tetra-methyl-4′-piperidinyl)propionylamide; 1,2-bis-(3,3,5,5-tetramethyl-2-oxo-piperazinyl)ethane; 4-oleoyloxy-2,2,6,6-tetramethylpiperidine; and combinations thereof.

UV absorbers, such as benzotriazoles or benzopheones, may also be excluded from the polymer composition of the present disclosure. Such benzotriazoles may include, for instance, 2-(2-hydroxyphenyl)benzotriazoles, such as 2-(2-hydroxy-5-methylphenyl)benzotriazole; 2-(2-hydroxy-5-tert-octylphenyl)benzotriazole (Cyasorb® UV 5411 from Cytec); 2-(2-hydroxy-3,5-di tert-butylphenyl)-5-chlorobenzo-triazole; 2-(2-hydroxy-3-tert-butyl-5-methylphenyl)-5-chlorobenzotriazole; 2-(2-hydroxy-3,5-dicumylphenyl)benzotriazole; 2,2′-methylenebis(4-tert-octyl-6-benzo-triazolylphenol); polyethylene glycol ester of 2-(2-hydroxy-3-tert-butyl-5-carboxyphenyl)benzotriazole; 2-[2-hydroxy-3-(2-acryloyloxyethyl)-5-methylphenyl]-benzotriazole; 2-[2-hydroxy-3-(2-methacryloyloxyethyl)-5-tert-butylphenyl]benzotriazole; 2-[2-hydroxy-3-(2-methacryloyloxyethyl)-5-tert-octylphenyl]benzotriazole; 2-[2-hydroxy-3-(2-methacryloyloxyethyl)-5-tert-butylphenyl]-5-chlorobenzotriazole; 2-[2-hydroxy-5-(2-methacryloyloxyethyl)phenyl]benzotriazole; 2-[2-hydroxy-3-tert-butyl-5-(2-methacryloyloxyethyl)phenyl]benzotriazole; 2-[2-hydroxy-3-tert-amyl-5-(2-methacryloyloxyethyl)phenyl]benzotriazole; 2-[2-hydroxy-3-tert-butyl-5-(3-methacryloyloxypropyl)phenyl]-5-chlorobenzotriazole; 2-[2-hydroxy-4-(2-methacryloyloxymethyl)phenyl]benzotriazole; 2-[2-hydroxy-4-(3-methacryloyloxy-2-hydroxypropyl)phenyl]benzotriazole; 2-[2-hydroxy-4-(3-methacryloyloxypropyl)phenyl]benzotriazole; and combinations thereof.

Benzophenone light stabilizers may include 2-hydroxy-4-dodecyloxybenzophenone; 2,4-dihydroxybenzophenone; 2-(4-benzoyl-3-hydroxyphenoxy)ethyl acrylate (Cyasorb® UV 209 from Cytec); 2-hydroxy-4-n-octyloxy)benzophenone (Cyasorb® 531 from Cytec); 2,2′-dihydroxy-4-(octyloxy)benzophenone (Cyasorb® UV 314 from Cytec); hexadecyl-3,5-bis-tert-butyl-4-hydroxybenzoate (Cyasorb® UV 2908 from Cytec); 2,2′-thiobis(4-tert-octylphenolato)-n-butylamine nickel(II) (Cyasorb® UV 1084 from Cytec); 3,5-di-tert-butyl-4-hydroxybenzoic acid, (2,4-di-tert-butylphenyl)ester (Cyasorb® 712 from Cytec); 4,4′-dimethoxy-2,2′-dihydroxybenzophenone (Cyasorb® UV 12 from Cytec); and combinations thereof.

The polymer composition of the present disclosure may contain various other additives and ingredients, particularly additives and ingredients that are safe for food contact and/or medical applications. For example, in one embodiment, one or more coloring agents can be incorporated into the polymer composition. The one or more coloring agents can be present in the composition in an amount less than about 2% by weight and generally in an amount greater than about 0.1% by weight. Colorants that may be used include any desired inorganic pigments, such as titanium dioxide, ultramarine blue, cobalt blue, and other organic pigments and dyes. Other colorants include carbon black or various other polymer-soluble dyes.

The compositions of the present disclosure can be compounded and formed into a polymer article using any technique known in the art. For instance, the respective composition can be intensively mixed to form a substantially homogeneous blend. The blend can be melt kneaded at an elevated temperature, such as a temperature that is higher than the melting point of the polymer utilized in the polymer composition but lower than the degradation temperature. Alternatively, the respective composition can be melted and mixed together in a conventional single or twin screw extruder. Preferably, the melt mixing is carried out at a temperature ranging from 150 to 300° C., such as from 200 to 280° C., such as from 220 to 270° C. or 240 to 260° C. However, such processing should be conducted for each respective composition at a desired temperature to minimize any polymer degradation.

After extrusion, the compositions may be formed into pellets. The pellets can be molded into polymer articles by techniques known in the art such as injection molding, thermoforming, blow molding, and the like.

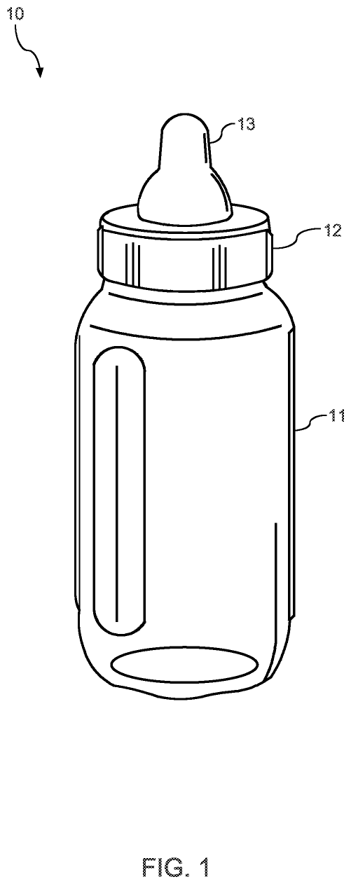

In one embodiment, the polymer composition of the present disclosure can be used to produce at least one component of a food contact article. For example, referring to FIG. 1, a baby bottle 10 is shown. The baby bottle 10 includes a container 11 attached to a nipple 13 by a collar 12. In one embodiment, the polymer composition of the present disclosure can be used to produce the collar 12 or a component of the collar 12.

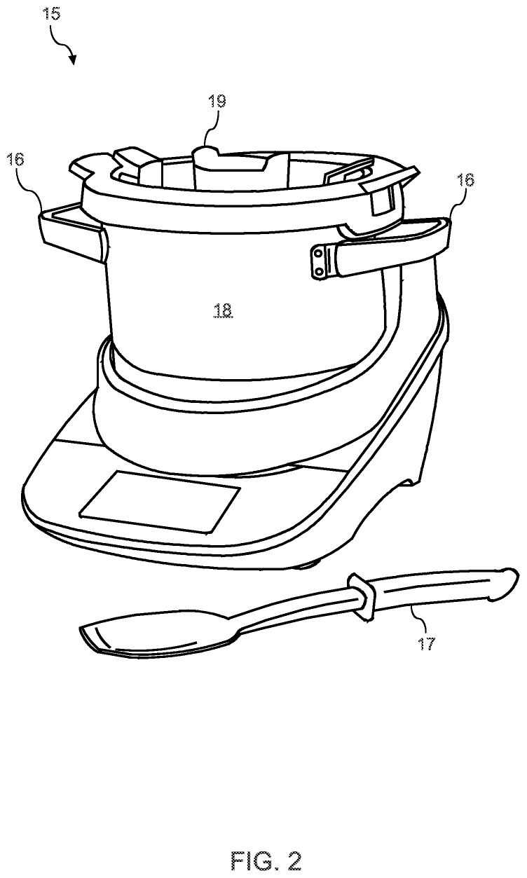

In an alternative embodiment, the polymer composition of the present disclosure can be used to produce one or more components of a consumer appliance or cooking apparatus. For instance, a cooking device 15 is shown in FIG. 2. The cooking device 15 includes a cooking pot 18 that meets with a lid 19. The cooking device 15 further includes handles 16. In one embodiment, the polymer composition of the present disclosure can be used to produce at least one component of the handle 16. For example, the handle 16 can be formed from the polymer composition of the present disclosure that is then overmolded with an elastomeric material that is also formulated for food contact applications. The elastomeric material, for instance, can be a thermoplastic polyurethane elastomer, a silicone elastomer, or a copolyester elastomer.

In one embodiment, the elastomeric component is formed from a copolyester elastomer. For example, in one embodiment, the elastomeric material may contain a segmented thermoplastic copolyester. The thermoplastic polyester elastomer, for example, may comprise a multi-block copolymer. Useful segmented thermoplastic copolyester elastomers include a multiplicity of recurring long chain ester units and short chain ester units joined head to tail through ester linkages. The long chain units can be represented by the formula

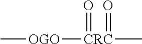

and the short chain units can be represented by the formula

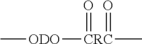

where G is a divalent radical remaining after the removal of the terminal hydroxyl groups from a long chain polymeric glycol having a number average molecular weight in the range from about 600 to 6,000 and a melting point below about 55° C., R is a hydrocarbon radical remaining after removal of the carboxyl groups from dicarboxylic acid having a molecular weight less than about 300, and D is a divalent radical remaining after removal of hydroxyl groups from low molecular weight diols having a molecular weight less than about 250.

The short chain ester units in the copolyetherester provide about 15 to 95% of the weight of the copolyetherester, and about 50 to 100% of the short chain ester units in the copolyetherester are identical.

In one particular embodiment, the polyester thermoplastic elastomer has the following formula: -[4GT]ₓ[BT]_{y}, wherein 4G is butylene glycol, such as 1,4-butane diol, B is poly(tetramethylene ether glycol) and T is terephthalate, and wherein x is from about 0.60 to about 0.99 and y is from about 0.01 to about 0.40.

In one aspect, the thermoplastic polyester elastomer can be a block copolymer of polybutylene terephthalate and polyether segments and/or dimerdiol segments and can have a structure as follows:

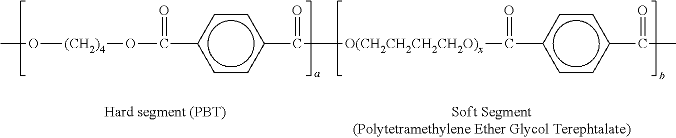

wherein a and b are integers and can vary from 2 to 50,000, such as from about 2 to about 10,000. The ratio between hard and soft segments in the block copolymer as described above can be varied in order to vary the properties of the elastomer.

In one aspect, the elastomeric material can contain a copolyester elastomer comprising a block copolymer containing polybutylene terephthalate segments and polytetramethylene ether glycol terephthalate segments.

In one aspect, the density of the polyester elastomer as indicated above can be from about 1.05 g/cm³ to about 1.15 g/cm³, such as from about 1.08 g/cm³ to about 1.2 g/cm³.

In one aspect, the copolyester elastomer can have a Shore D hardness of less than about 100, such as less than about 90, such as less than about 80, such as less than about 70, such as less than about 60, such as less than about 50, such as less than about 40. The Shore D hardness of the elastomer can generally be greater than about 10, such as greater than about 15, such as greater than about 20, such as greater than about 25.

The elastomeric material can be applied to the polymer component made from the polyester composition using any suitable method or technique. In one embodiment, for instance, the elastomeric material is overmolded onto the surface of the polymer component to form the elastomeric component. For example, the polymer component can first be injection molded and prior to cooling, can be overmolded with the elastomeric material also using an injection molding process.

As shown in FIG. 2, the cooking apparatus 15 can be used in conjunction with a spatula 17. The spatula 17 can also be made from the polymer composition of the present disclosure.

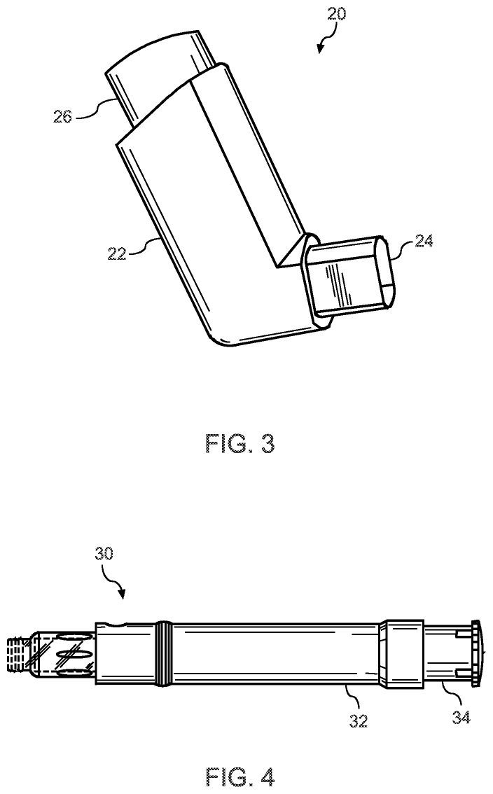

In other embodiments, the polymer composition of the present disclosure can be used to produce at least one component of a medical device. For instance, referring to FIG. 3, an inhaler 20 is shown. The inhaler 20 includes a housing 22 attached to a mouthpiece 24. In operative association with the housing 22 is a plunger 26 for receiving a canister containing a composition to be inhaled. The composition may comprise a spray or a powder. The inhaler 20 can include a first sliding member in operative association with a second sliding member. For instance, in certain embodiments, the housing 22 may comprise the first sliding member while the plunger 26 may comprise the second sliding member. Alternatively, the first sliding member may comprise the housing 22 and the second sliding member may comprise the mouthpiece 24. In still another embodiment, an internal sliding member may be contained within the housing 22 that slides relative to the housing.

During use, the inhaler 20 administers metered doses of a medication, such as an asthma medication to a patient. The asthma medication may be suspended or dissolved in a propellant or may be contained in a powder. When a patient actuates the inhaler to breathe in the medication, a valve opens allowing the medication to exit the mouthpiece.

In another embodiment, the polymer composition of the present disclosure can be used to produce one or more components of an injector, particularly an autoinjector. For example, a medical injector 30 is shown in FIG. 4. The medical injector 30 includes a housing 32 in operative association with a plunger 34. The housing 32 or first sliding member may slide relative to the plunger 34 or second sliding member. The medical injector 30 may be spring loaded. The medical injector 30 is for injecting a drug into a patient, typically into the thigh or the buttocks. The medical injector can be needleless or may contain a needle. When containing a needle, the needle tip is typically shielded within the housing prior to injection. Needleless injectors, on the other hand, can contain a cylinder of pressurized gas that propels a medication through the skin without the use of a needle.

The present disclosure may be better understood with reference to the following examples.

### EXAMPLES

The following examples are given below by way of illustration and not by way of limitation. The following experiments were conducted in order to show some of the benefits and advantages of the present invention.

### Example No. 1

Various polymer compositions containing glass fibers were formulated and tested for physical properties. Each of the compositions contained a polybutylene terephthalate polymer, a talc nucleant, and glass fibers in an amount of 30% by weight.

Four different compositions were formulated. The first two compositions were formulated and tested for purposes of comparison. Sample Nos. 1 and 2 both contained glass fibers in which the sizing composition applied to the fibers contained conventional amounts of bisphenol-A. Sample No. 3 made in accordance with the present disclosure contained glass fibers in which the sizing composition of the fibers contained minor amounts of bisphenol-A. The glass fibers contained in Sample No. 4 were bisphenol-A free.

The following compositions were formulated and tested:

| | Sample | Sample | Sample | Sample |
|---|---|---|---|---|
| Component | No. 1 | No. 2 | No. 3 | No. 4 |
| Polybutylene terephthalate | 70.0 | 69.7 | 69.85 | |
| (62 cm³/10 min) | | | | |
| Polybutylene terephthalate | | | | 69.85 |
| (40 cm³/10 min) | | | | |
| Glass fiber (diameter 13 | 30 | 30 | | |
| microns) | | | | |
| Glass fiber (diameter 10.5 | | | 30 | |
| microns) | | | | |
| Glass fiber (diameter 10 | | | | 30 |
| microns) | | | | |
| Talc nucleant | | | 0.15 | 0.15 |
| Ethylene bis stearamide wax | | 0.3 | | |

The components of each respective composition were mixed together and compounded using a ZSK 25MC (Werner & Pfleiderer, Germany) twin screw extruder. The screw configuration with kneading elements was chosen so that effective thorough mixing of the components took place. The compositions were extruded and pelletized. The pellets were dried for 4 hours at 120° C. and then injection molded.

The compositions were tested for physical properties. Tensile properties were tested according to ISO Test 527:2012. Notched and unnotched Charpy impact strength was tested according to ISO Test 179-1:2010. The test was run using a Type A notch (0.25 mm base radius) and Type 1 specimen size (length of 80 mm, width of 10 mm, and thickness of 4 mm). The test was conducted at a temperature of 2300. The following results were obtained:

| | Sample | Sample | Sample | Sample |
|---|---|---|---|---|
| Property | No. 1 | No. 2 | No. 3 | No. 4 |
| Tensile modulus (MPa) (1 | 9200 | 9200 | 10,000 | 10,350 |
| mm/min) | | | | |
| Break stress (MPa) (50 | 130 | 130 | 154 | 158 |
| mm/min) | | | | |
| Break strain (%) (50 mm/min) | 2.5 | 2.5 | 2.5 | 2.7 |
| Charpy notched @ | 8.5 | 8.5 | 9.5 | 9.5 |
| 23° C. (kJ/m²) | | | | |
| Charpy unnotched @ | 46 | 46 | 60 | 60 |
| 23° C. (kJ/m²) | | | | |
| Melt flow rate (250° C./2.16 | 16 | 16 | 12 | 9 |
| kg) (cm³/10 min) | | | | |

As shown above, Sample Nos. 3 and 4 made according to the present disclosure showed dramatically improved strength and impact resistance properties in comparison to Sample Nos. 1 and 2.

Sample Nos. 3 and 4 were tested for free bisphenol-A using the extraction method described previously. Sample No. 3 was found to contain 1.54 ppm of free bisphenol-A. The amount of free bisphenol-A in Sample No. 4 was below detectable limits, i.e. less than 0.1 ppm.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A food and medical grade polymer composition comprising:
a polyester polymer comprising a polybutylene terephthalate polymer, the polyester polymer being present in the composition in an amount greater than about 40% by weight;
a mineral nucleant being present in the composition in an amount less than about 0.8% by weight; and
reinforcing fibers comprising glass fibers, the reinforcing fibers being present in the polymer composition in an amount from about 3% by weight to about 50% by weight, the reinforcing fibers containing free bisphenol-A in an amount of less than about 2 ppm.

2. A food and medical grade polymer composition as defined in claim 1, wherein the polymer composition does not contain a hindered phenolic antioxidant.

3. A food and medical grade polymer composition as defined in claim 1 or 2, wherein the polymer composition does not contain a diphosphite stabilizer.

4. A food and medical grade polymer composition as defined in claim 1, wherein the polymer composition does not contain a wax.

5. A food and medical grade polymer composition as defined in claim 1, wherein the polybutylene terephthalate polymer, the glass fibers, and the mineral nucleant account for at least about 95% by weight.

6. A food and medical grade polymer composition as defined in claim 1, wherein the glass fibers have an average diameter of less than about 17 microns and greater than about 3 microns.

7. A food and medical grade polymer composition as defined in claim 1, wherein the mineral nucleant comprises talc particles, the talc particles being present in the polymer composition in an amount from about 0.01% by weight to about 0.4% by weight.

8. A food and medical grade polymer composition as defined in claim 1, where every component contained in the polymer composition complies with the United States Food and Drug Administration Standards listed in 21 CFR 177.

9. A food and medical grade polymer composition as defined in claim 1, wherein the glass fibers and/or the polymer composition contain free bisphenol-A in an amount less than about 100 ppb.

10. A food and medical grade polymer composition as defined in claim 1, wherein the polybutylene terephthalate polymer has a melt flow rate of from about 50 g/10 min to about 80 g/10 min.

11. A food and medical grade polymer composition as defined in claim 1, wherein the polybutylene terephthalate polymer has a melt flow rate of from about 25 g/10 min to about 70 g/10 min.

12. A food and medical grade polymer composition as defined in claim 1, wherein the composition displays a tensile strength at break of greater than about 135 MPa, displays a tensile modulus of greater than about 9,500 MPa, and displays a Charpy notched impact strength of greater than about 9 kJ/m².

13. A food and medical grade polymer composition comprising:
a polyester polymer comprising a polybutylene terephthalate polymer, the polyester polymer being present in the composition in an amount greater than about 40% by weight;
a mineral nucleant being present in the composition in an amount less than about 0.8% by weight; and
reinforcing fibers comprising glass fibers, the reinforcing fibers being present in the polymer composition in an amount from about 3% by weight to about 50% by weight, the reinforcing fibers having an average diameter of less than about 11 microns and greater than about 5 microns, the glass fibers being present in the polymer composition in an amount from about 15% by weight to about 40% by weight.

14. A food and medical grade polymer composition as defined in claim 13, wherein the glass fibers do not contain free bisphenol-A.

15. A food and medical grade polymer composition as defined in claim 13, wherein the polymer composition does not contain a hindered phenolic antioxidant, a diphosphite stabilizer, or a wax, and wherein the polybutylene terephthalate polymer, the glass fibers, and the mineral nucleant account for at least about 95% by weight, such as at least about 97% by weight, such as at least about 99% by weight of the polymer composition.

16. A food and medical grade polymer composition as defined in claim 13, wherein the composition displays a tensile strength at break of greater than about 135 MPa, such as greater than about 140 MPa, such as greater than about 145 MPa, such as greater than about 150 MPa, displays a tensile modulus of greater than about 9,500 MPa, such as greater than about 9,750 MPa, such as greater than about 9,900 MPa, and displays a Charpy notched impact strength of greater than about 9 kJ/m², such as greater than about 9.2 kJ/m².

17. A molded article made from the polymer composition as defined in claim 1.

18. A baby bottle in which at least one component of the bottle comprises the molded article as defined in claim 17.

19. A medical device in which at least one component of the medical device comprises the molded article as defined in claim 17 and wherein the medical device comprises an inhaler or an injector.

20. A method for determining the amount of free bisphenol-A in a sample comprising:
combining a sample with methanol;
extracting the sample and methanol combination via Soxhlet extraction at a temperature of from about 50° C. to about 60° C. for 48 hours to form a slurry;
filtering the slurry and drying to form a residue;
suspending the residue in methanol to form a suspension; and
conducting high-resolution mass spectrometry on the suspension to determine an amount of free bisphenol-A.
